(19) United States
(12) Patent Application Publication
(10) Pub. No.: US 2009/0264510 A1
(43) Pub. Date: Oct. 22, 2009

(54) Double helical oligonucleotides interfering with mRNA used as effective anticancer agents

(76) Inventors: Maciej Wieczorek, Lomianki (PL); Joanna Wietrzyk, Wroclaw (PL); Anna Nasulewicz, Skierniewice (PL); Katarzyna Szczaurska, Kluczbork (PL); Jan Piotr Guzenda, Lodz (PL); Monika Lamparska-Przyhysz, Warszawa (PL)

Correspondence Address: Paul G. Lunn, Esq., 14606 - 135th Ct. N.E., Woodinville, WA, 98072-4607, US

(21) Appl. No.: 12/221,063

(22) Filed: Jul. 30, 2008

Related U.S. Application Data
(63) Continuation-in-part of application No. PCT/PL07/00006, filed on Jan. 31, 2007.

(30) Foreign Application Priority Data
Jan. 31, 2006 (PL) P.378857

(51) Int. Cl. A61K 31/7088; C12Q 1/68; C07H 21/02

(52) U.S. Cl. 514/44 R; 435/6; 536/23.1

(57) ABSTRACT

The present invention relates to the application of double-helical oligonucleotides (siRNA) interfering with the mRNA of gene involved in carcinogenesis, particularly the Wnt1, Wnt2 or Her3 genes. Such oligonucleotides may be modified chemically, used in conjunction with viral and non-viral vectors such as lipid complexes. Such oligonucleotides exhibit unusual anti-proliferative properties against tumour cells and may be used in anti-tumour treatment.

### RELATED APPLICATIONS

This is a continuation-in-part of and claims benefit under 35 U.S.C. §120 of International Patent Application No. PCT/PL2007/000006 filed on Jan. 31, 2007, which claims benefit under 35 U.S.C. §119 of Polish Patent Application No. P.378857 filed on Jan. 31, 2006, the teachings of which are all incorporated herein in their entirety by reference.

### BACKGROUND

The teachings of all of the references, including websites, cited herein are incorporated herein in their entirety by reference. The present invention relates to the application of double-stranded oligonucleotides interfering with the mRNA transcribed from a gene involved in carcinogenesis, particularly the Wnt1, Wnt2 or Her3 gene, as novel anti-tumour agents.

RNA interference is a phenomenon based on the post-transcriptional gene silencing (PTGS) and is an excellent tool for the analysis of their function and role in many processes within an organism. This technique is of great importance in functional genomics, mapping of biochemical pathways, determination of pharmacological treatment directions and in gene therapy. PTGS was first described in plants (Napoli, C., C. Lemieux and R. Jorgensen. Introduction of a Chimeric Chalcone Synthase Gene into Petunia Results in Reversible Co-Suppression of Homologous Genes in trans. Plant Cell 2:279-289, 1990) In 1998, Andrew Fire and Craig Mello described RNAi for the first time in an animal, C. elegans (Fire, A. et al. 1998 Potent and specific genetic interference by double-stranded RNA in Caenorhabditis elegans. Nature 391, 806-810). Long, double-stranded RNA molecules induced post-transcriptional gene silencing.

However, the application of nucleotides this long also elicited an immune response (increased interferon levels) in mammalian cells and it was T. Tuschl, S M. Elbashir et al. who finally discovered that the application of short, double-stranded nucleotides (19-21 bp) does not induce an immune response (Elbashir, S. M., J Harborth, W. Lendeckel, A. Yalcin, K Weber and T. Tuschl. 2001. Duplexes of 21-nucleotide RNAs mediate RNA interference in cultured mammalian cells. Nature 411:494-498).

Gene silencing is based on double-stranded interfering nucleic, acid (iNA) preferably a double-stranded RNA (dsRNA) molecules, also called siRNA. RNAi is a response to cellular processes induced by dsRNA, which degrades homologous mRNA. Even a few copies of dsRNA may entirely destroy the transcripts for a given gene formed within a cell. The destruction of selected mRNA's through RNAi begins with the activation of RNAse III, which cleaves long hairpin loops of dsRNA or ssRNA fragments into double-stranded small interfering RNA (siRNA) 21-23 nucleotides long. siRNA's prepared earlier may be introduced into cells externally. Next, siRNA molecules bind to a nuclease complex forming a RISC(RNA induced silencing complex). Thanks to the helicase activity which is a part of the RISC, dsRNA is separated into single strands. The ssRNA molecules formed then anneal to complementary mRNA strands. The final stage of PTGS is the degradation of selected mRNA by RISC nucleases. In contrast to traditional methods, such as knockouts, gene silencing is quickly and easily performed, both in animal and in cell line models.

The authors of the present invention have performed intensive research and have determined that the silencing of expression of genes involved in carcinogenesis, e.g. gene Wnt1, using double-stranded oligonucleotides such as an iNA or an siRNA for this gene is an effective strategy for the inhibition of tumour cell proliferation.

Wnt1 is a secretory protein which binds the “frizzled” inter-membrane receptor and transmits a signal to cytoplasmatic phosphoproteins, which in turn downregulate the constitutively high activity of glycogen synthase kinase 3Beta (GSK-3Beta) (Polakis et al., Wnt signaling and cancer, Genes Dev. 2000 Aug. 1; 14(15):1837-51). The result of this is the stabilization and growth of Beta-catenin levels in the cell nucleus.

Wnt-1 overexpression has been noted in many types of tumours, including in cancers of the lung, colon and breast, sarcomas and tumours of the head and neck (Katoh et al. Expression and regulation of WNT1 in human cancer: up-regulation of WNT1 by beta-estradiol in MCF-7, In J Oncol, 2003 January; 22(1):209-12).

Anti-WNT-1 monoclonal antibodies are known. The application of such antibodies resulted in an increase of apoptosis, a decrease in tumour cell proliferation (H460 and MCF-7 lines), as well as inhibition of the take of transplantable murine lung cancer (H460) (Biao He, A Monoclonal Antibody against Wnt-1 Induces Apoptosis in Human Cancer Cells, Neoplasia, Vol. 6, No. 1, January/February 2004, pp. 7-14). In the above report, Biao He et al. also used chemically unmodified siRNA on a breast cancer line (MCF-7), resulting in an increased apoptosis rate in these cells.

Anti-WNT-1 monoclonal antibodies elicited apoptosis in sarcoma cells (A-204) (Iwao Mikami, Efficacy of Wnt-1 monoclonal antibody in sarcoma cells, BMC Cancer 2005, 5:53, 24 May 2005), and in NCI-H1703 and H28 lung cancer cells (Liang You, Inhibition of Wnt-1 Signaling Induces Apoptosis in β-Catenin-Deficient Mesothelioma Cells, Cancer Research 64, 3474-3478, May 15, 2004). This research also made use of chemically unmodified siRNA in MCF-7 breast cancer cells, and NCI-H1703 and H28 lung cancer cells, resulting in an increased apoptosis rate.

You et al. also used unmodified siRNA, which elicited apoptosis to a degree similar to monoclonal antibodies. Similar results of apoptosis induction were obtained in colon cancer cells (SW-480, HCT116) (He et al., Blockade of Wnt-1 signaling induces apoptosis in human colorectal cancer cells containing downstream mutations, Oncogene 2005, 24: 3054-3058). In a recent report, Fukutomi et al. (Hepatology 2005; 41:1096-105) indicated only an indirect effect of the siRNA silencing of WNT-1 on the proliferation of modified liver tumour cells. These experiments made use of liver cancer line cells expressing type C hepatitis virus core protein. Expression of type C hepatitis virus core protein was obtained through the transfection of these cells with vectors coding for said protein. The presence of this protein enhanced WNT-1 expression and cell proliferation. The application of siRNA specific for Wnt-1 in such cells caused the silencing of its expression and inhibited proliferation. This sort of experimental model, however, does not provide evidence which would allow one to hypothesize that a similar effect would be elicited in cells unmodified with the viral protein, upon the application of WNT-1 specific siRNA.

International Patent Application Publication No. WO2004032838 describes a method of inhibiting tumour cell proliferation based on the contact of a cell with a compound which blocks the interaction of WNT with its receptor. As an example of such inhibition, a monoclonal antibody against the WNT-1 protein was used. This patent application also describes the occurrence of apoptosis in the cells of many tumour lines following the application of siRNA for the WNT-1 protein. None of the above publications describes any effect of oligonucleotides which activate the siRNA mechanism in the inhibition of the proliferation of unmodified tumour cells, nor is such an effect known.

The elimination of cells through apoptosis is not a sufficient mechanism for enhancement of anti-tumour activity, because in many tumour types this mechanism is disrupted or inhibited. Among other factors, this is connected with a series of mutations in the p53 gene, which is responsible for regulation of this process. The inefficacy of this process may also be tied in with the absence of proapoptotic proteins such as Bax or Bid in many types of tumours, or the increased expression of apoptosis inhibitors such as Bcl-2. Only the inhibition of tumour take and/or tumour cell proliferation can be evidence of anti-tumour activity.

Experiments on modified cells do not facilitate the prediction of the behaviour of natural, unmodified cells occurring in tumours. The application of monoclonal antibodies as a potential treatment entails a considerable risk of eliciting an immune response in living organisms. Additionally, monoclonal antibodies are very expensive and their production does not guarantee a repeatable response in individual recipients, since genetically modified organisms are used in their manufacture.

Thus, there exists a real need to find new, effective treatments which would exhibit anti-tumour properties but which would not elicit immune responses. Such drugs should be simple and inexpensive to manufacture, preferably using a reproducible technological process.

### SUMMARY OF THE DISCLOSURE

The present invention relates to the use siRNA against mRNA transcribed by genes involved in carcinogenesis. An example of such an siRNA is one that targets the mRNA of the oncogene Wnt1 gene. An siRNA directed to tumour cell lines expressing the Wnt1 results in a strong inhibition of tumour cell proliferation. This inhibition is dose-dependent. The present invention thus successfully delivers a solution to the problem of tumour treatment through the inhibition of tumour cell growth, using the RNA interference mechanism to degrade the mRNA of the gene involved in carcinogenesis, e.g. gene coding WNT-1.

The present invention provides methods of inducing apoptosis or inhibiting growth of a cancer cell as well the method for obtaining the oligonucleotide useful as an effective anticancer agent. Furthermore, the application of the present invention entails a very limited danger of eliciting an immune response in treated patients. The production of double-helical oligonucleotides is a reproducible process and is simple to perform using standard equipment, the so-called RNA synthesizers. Such oligonucleotides may be designed according to one of many algorithms described to date, such as the one indicated in Example 1.

The sequence of an mRNA gene of interest can be obtained from a database, for example GenBank, and the NCBI Reference Sequence should be chosen. The second structure of the mRNA target sequence can be designed using computer folding algorithm. siRNAs against a chosen mRNA sequence can be generated in silico using known algorithms. There are many algorithms available on-line that are used to design siRNAs against a particular mRNA sequence. These algorithms in general are based on similar equations but there are subtle differences among them. Most of algorithms are based on Tuschl rules of siRNA design but some of them additionally use also Reynolds rules (Reynolds A, Leake D, Boese Q, Scaringe S, Marshall W S, Khvorova A. Rational siRNA design for RNA interference. Nat Biotechnol. 2004 March; 22(3):326-330). It is known that you have to verify siRNAs generated by one of the algorithms with another algorithm. That is why in our method we use different algorithms based on different equations. The Tuschl Rules are:

Select targeted region from a given cDNA sequence beginning 50-100 nt downstream of start condon
2. First search for 23-nt sequence motif AA(N₁₉). If no suitable sequence is found, then,
3. Search for 23-nt sequence motif NA(N₂₁) and convert the 3′ end of the sense siRNA to TT
4. Or search for NAR(N₁₇)YNN
5. Target sequence should have a GC content of around 50%, wherein

### A=Adenine; T=Thymine; R=Adenine or Guanine (Purines); Y=Thymine or Cytosine (Pyrimidines); N=Any.

Some algorithms also analyze the thermodynamic stability of siRNAs. The distribution of free energy through siRNA molecule is a very important factor in describing the potential of a given sequence. This feature is very important in recognition of the guide strand by RISC because RISC recognizes the 5′ end of a strand that will be incorporated into RISC, and will serve as the guide strand. It is known that the 5′ end of the antisense strand should be less stable than the 3′ end, so the free energy at the 5′ end should be higher than at the 3′ end.

Relying on these rules, there should be a difference in GC content between the 5′ and 3′ ends. More GC pairs are preferred at a 3′ end of antisense strand. Also the total content of GC in an siRNA is important for the thermodynamic stability of siRNA and its potential. In functional siRNA, GC content should be between 30%-60%; this will ensure that a designed duplex will not be too stable to be unwound and will be stable enough to avoid self-unwinding in cytoplasm.

In thermodynamics analysis it is also recommended to design siRNA with a low stability at position 10 of antisense strand. This position is a cleavage site so there should not be formed a strong duplex between guide strand and target mRNA, U base is recommended in this position. Another factor that should be taken into consideration during siRNA designing is to target secondary structure accessibility. This factor describes probability of a single stranded motif in target region in mRNA molecule. In the cytoplasm mRNA never exists as a single strand, its secondary structure is rich in hairpins, loops and other structures which are results of partial paring between bases in given mRNA molecule.

One of the greatest problems in designing siRNA is to avoid potential “off-target” effect. This effect occurs if particular siRNA targets not only desired mRNA but other mRNAs as well. In this case there are also many algorithms like blast or clustal which can predict possible interactions with any known transcript.

1. The sequence of an mRNA gene of interest was obtained from a database, for example GenBank, and the NCBI Reference Sequence was chosen. siRNAs against chosen mRNA sequence were generated in silico using known algorithms.

The designed sequences were ranked according to total filtering score based on following rules:

a) Frequency among algorithms.
This is the value that describes how many algorithms chose a particular siRNA. This value is described by equation:

a=1*number of algorithms

b) Single stranded region probability
This is the value that describes probability that there is a single stranded motif in target region of particular mRNA molecule. This value is calculated using computer folding algorithm or it can be calculated pursuant to equation:

$$b = \frac{Mss}{Mt}$$

where:
Mss—number of secondary structures in which there is a single stranded motif in a target region
Mt—total number of secondary structures predicted

c) Complementary to other mRNA sequences
This is the value that describes possibility of “off-target” effect. This value is described by equation:

c=−2*number of molecules

d) Free energy of the antisense strand 5′ end
This is the value that describes the stability of the 5′ end of the antisense strand of the siRNA. This value is calculated based on RNA thermodynamics parameters.

e) Free energy of the antisense strand 3′ end
This is the value that describes the stability of the 3′ end of the antisense strand of the siRNA. This value is calculated based on RNA thermodynamics parameters.

f) Free energy at 10 position of the antisense strand
This is the value that describes a stability of a cleavage site. This value is calculated based on RNA thermodynamics parameters.

g) GC content
This is the value that describes the stability of the siRNA molecule. This value is calculated being based on equation:

$$g = \frac{{N}_{G}}{{N}_{T}} * 100 \% ,$$

where:
N_{G}—number of G bases in both strands
N_{T}—total number of bases in antisense strand

For further analyses the best fifteen siRNAs have been chosen.

Then screenings for inhibition of proliferation, decrease in mRNA and protein levels were performed. The experiments were enforced by transfection efficiency greater or equal to 80%.

The inhibition score for each sequence was evaluated by factor s:

$$s = 1 - ( \frac{Rs - Rm}{Rc - Rm} ) ,$$

where:
   Rs—the result of a measurement of a probe with siRNA
   Rm—the result of a measurement of a blank probe
   Rc—the result of a measurement of a probe with control

Scores:
i. 0 if s lower than 0.50
ii. 1 if s value 0.51-0.60
iii. 2 if s value 0.61-0.70
iv. 3 if s value 0.71-0.80
v. 4 if s value 0.81-0.90
vi. 5 if s value 0.91-1.00

Then sequences were ranked by the inhibition score.
For further analyses siRNAs which rank better or equal to 50% of the best sequence have been chosen.

The decrease in mRNA level score for each sequence was evaluated by factor r:

$$r = 100 - ( \frac{Es}{Ec} * 100 ) ,$$

where:
Es—relative expression of target gene in probe with siRNA
Ec—relative expression of target gene in probe with control

Scores:
0 ifs lower than 50
1 if s value 51-60
2 if s value 61-70
3 if s value 71-80
4 if s value 81-90
5 if s value 91-100

Then sequences were ranked by the decrease in mRNA level score.

The decrease in protein level score for each sequence was evaluated by factor t:

$$t = 100 - ( \frac{Ps}{Pc} * 100 )$$

where:
Ps—protein level in probe with siRNA
Pc—protein level in probe with control

Scores:
0 if s lower than 50
1 if s value 51-60
2 if s value 61-70
3 if s value 71-80
4 if s value 81-90
5 if s value 91-100

Then sequences were ranked by the decrease in protein level score.

All sequences were characterized by final screening factor z:

$$z = ( \frac{b + c}{2} ) * a$$

where:
a—rank by the inhibition score
b—rank by the decrease in mRNA level score
c—rank by the decrease in protein level score

Next siRNAs with z factor better or equal to 50% of the best sequence, but not more than 3 were analyzed according to the cell death mechanism. Sequences were ranked by:

number of alive cells

−1*% of necrotic cells

+1*% of early apoptotic cells

+2*% of apoptotic cells

Next dose-response effect was evaluated.

the lowest dose by which over 65% of silencing had been achieved was evaluated,

the longest period of time with effect still observed was evaluated.

Moreover, to limit the immune response, it is preferable that the oligonucleotides be no more than 30 bp long, and preferentially be 21-23 bp long. Sense and antisense oligonucleotides may be symmetrical or not, meaning that i.e. 2 terminal nucleotides may be unhybridized, thus forming sticky ends. In order to enhance their thermal and enzymatic stability, pharmacokinetic, bioavailability and cellular uptake properties the oligonucleotides may be modified chemically. Chemical modifications may pertain to phosphates, ribose or the nucleases themselves. Said chemical modifications may pertain to only selected nucleotides, i.e. terminal or median, or the entire oligonucleotide.

The oligonucleotides may be delivered to tumour cells both by themselves, without vectors, as well as with a vector, both viral and non-viral. Adenoviruses or adeno-like viruses are examples of viral vectors, which facilitate the continual expression of the oligonucleotide following introduction into tumour cells. Non-viral vectors used to introduce oligonucleotides into cells are lipid capsules, lipid complexes or other vectors prolonging their half-lives in a living organism and/or absorption into cells. As a result of use of present invention, considerable inhibition of tumour cell proliferation is achieved.

Although the examples and descriptions presented below illustrate the nature of the present invention and include examples to illustrate it, it is understood that a practical embodiment of the present invention encompasses all normal changes, adaptations, modifications, deletions from or additions to the procedures described, being a part of the below claims and equivalents.

### BRIEF DESCRIPTION OF THE DRAWINGS

FIG. 1 Percent of proliferation inhibition after transfection of MCF-7 cells with sixteen siRNAs sequences against Wnt1 gene in concentration 50 nM for 48 h with respect to untreated cells. Cells viability was measured using MTS test.

FIG. 2 Decrease of Wnt1 protein level in MCF-7 cells after transfection with specific siRNA to Wnt1. a) Expression of Wnt1 and actin in MCF-7 cell line 48 h after siRNA against Wnt1 treatment. b) Percent of cells expressing Wnt1 24 h and 72 h after W13, W15 and WP sequences treatment.

FIG. 3 Cell cycle analysis after treatment with siRNA against Wnt1. a) Cytograms showing DNA content and cell size of MCF-7 cells 72 h after transfection with W15 and WP sequences. b) Histograms presenting cell cycle of MCF-7 cells 72 h after transfection with W15 and WP sequences.

FIG. 4 Apoptosis after treatment with W15 sequence. a) Activity of caspases 3 and 7. b) Morphological changes of MCF-7 cells after treatment with W15 sequence.

FIG. 5 Apoptosis analysis using Annexin V and propidium iodide staining of MCF-7 cells after Wnt1 siRNA. a) Cytograms presenting morphology of MCF-7 cells 72 h after transfection with W15 and WP sequences. b) Cytograms showing number of cells in early, late phase of apoptosis and necrosis 72 h after transfection with W15 and WP sequences.

FIG. 6 Wnt1 siRNA induces apoptosis triggered by decrease in protein level of Wnt1 in MCF-7 cells. a) Cytograms presenting DNA content and expression of Wnt1. b) Histograms showing number of cells with high and low expression of Wnt1.

FIG. 7 siRNAs ranking results. Eight siRNAs passed inhibition score ranking (bold), two sequences passed z score ranking (bold, red). All factors for sequence WP (sequence from literature) for comparison were analyzed.

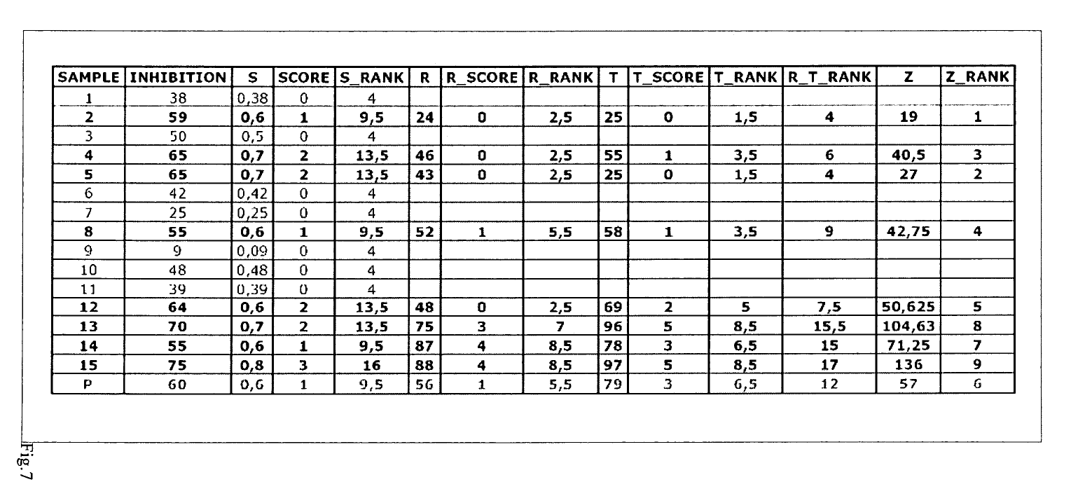

### DETAILED DESCRIPTION

### Materials and Methods

### Cell Culture

Human breast cancer cell line MCF-7 was obtained from the American Type Culture Collection (Rockville, Md., USA). Cell cultures were maintained in DMEM supplemented with 10% (v/v) fetal calf serum (FCS), 50 μg/ml gentamycin, 2.5 μg/mL fungizone, 50 UI/mL penicillin and 50 μg/ml streptomycin (Invitrogen Carlsbad, Calif. USA) in an atmosphere of 5% CO2/95% humidified air at 37° C., and routinely subcultured every 2 or 3 days.

### Cell Proliferation Analysis

For proliferation tests MCF-7 cells were plated in Opti-MEM (Invitrogen) at 7×103 cells per well in 96-well plates one day before experiments. The next day the MCF-7 cells were transfected with fifteen siRNAs sequences specific to Wnt1 mRNA and scrambled siRNA sequence (control) in at a concentration of 50 nM for 48 h using Lipofectamine RNAi MAX (Invitrogen) according to manufacturer's protocol. siCONTROL TOX (Dharmacon, Chicago, Ill. USA) was used as a control of transfection efficiency. After 48 h of experiment proliferation inhibition was measured using MTS test (Promega, Madison, Wis. USA).

Western Blot Analysis

Reagents for Western blotting were purchased from BioRad (Hercules, Calif. USA), anti-Wnt1 antibody was from Zymed Invitrogen, anti-actin, anti-phosphor-beta-catenin, anti-c-myc and anti-cyclin D1 were from Santa Cruz Biotechnology (Santa Cruz, Calif. USA), anti-cleaved PARP antibody was from Cell Signaling (Danvers, Mass., USA). Western blotting detection reagents was from Roche Diagnostics (Indianapolis, Ind., USA) and Light Film BioMax was from Kodak (Rochester, N.Y. USA)

On the day before the experiment, 250×103 cells were cultured in Opti-MEM in sterile 25 cm2 conical flasks to 60% confluence. To knock-down the Wnt1 gene, medium was removed and replaced with the transfection medium containing siRNAs, which passed the inhibition score ranking. After 48 h the cultured cells were harvested by trypsinization and centrifuged at 2000 g, for 5 min, at 4° C. and the cells pellet was suspended in ice-cold phosphate buffered saline (PBS). After second centrifugation the supernatant was removed and the cell pellet was re-suspended in 0.5 mL Total Lysis Buffer RIPA (Santa Cruz Biotechnology, Santa Cruz, Calif., USA), and incubated at 4° C. for 30 min. The cells suspended in the buffer were centrifuged at 9000 g, 10 min, at 4° C., then the supernatant (containing the total protein fraction) was carefully removed and passed six times through a 20-gauge syringe needle.

The lysates were mixed 1:2 (v/v) with Laemmli sample buffer (BioRad) containing 2.5% 2-mercaptoethanol and boiled for 3 min. Samples containing identical quantities of proteins were subjected to SDS-PAGE (12% gel) together with a Kaleidoscope Marker (BioRad). The electrophoresis was run for 1 hour at 100 V using a Mini Protean III cell (BioRad,). After electrophoresis the separated proteins were electroblotted on a PVDF membrane (Biorad) for 70 min at 110 V using the Mini Protean III. The membranes were blocked overnight with 5% w/v solution of non-fat powdered milk in TBST (pH 7.5). The following day the membranes were rinsed three times for 10 min in TBST, at room temperature, and then incubated for 1 hour at room temperature with the primary antibodies diluted 1:200. The membranes were then rinsed four times for 10 min in TBST and incubated with diluted 1:2000 secondary antibodies conjugated with horseradish peroxidase (Sigma Aldrich, St. Louis, USA) for another 1 h at room temperature. Finally, the membranes were rinsed three times for 10 min in TBST, and labelled proteins were visualized using the LumiLight (Roche) Western blotting detection reagent on a high performance chemiluminescence BioMAX light film (Kodak). The image on light film was then analyzed with a Kodak Edas System and the integrated optical density (IOD) was measured.

Real Time-PCR

On the day before the experiment, 250×103 cells were cultured in Opti-MEM in sterile 25 cm2 conical flasks to 60% confluence. To knock-down the Wnt1 gene, medium was removed and replaced with the transfection medium with siRNAs, which past the inhibition score ranking. After 48 h the cultured cells were harvested by trypsinization and centrifuged at 2000 g, for 5 min, at 4° C. and the cells pellet was suspended in ice-cold PBS. Then cells were lysed by adding 1 ml of TRIZOL Reagent (Invitrogen) and passed several times through a pipette. After that lysate was incubated for 5 minutes at room temperature. Next 0.2 mL of chloroform per 1 mL of TRIZOL was added and samples were incubated at room temperature for 10 min. Next samples were centrifuged at >12,000 g for 15 min at 4° C. Then the aqueous phase was transferred to a fresh tube and RNA was precipitated from the aqueous phase by mixing with isopropyl alcohol and incubated at room temperature for 10 min and centrifuged at >12,000 g for 10 min at 4° C. The RNA was washed once with 1 mL 75% ice-cold ethanol. Samples were mixed by vortexing and centrifuged at >12,000 g for 5 min at 4° C. At the end of the procedure, the RNA pellet was dried (air-dry for 5-10 min). At the end RNA was dissolved in proper volume of RNase-free water.

Isolated RNA was transcribed to cDNA using ImProm-II Reverse Transcriptase kit (Promega), according to manufacturer's protocol. Changes in mRNA expression of target genes were measured using Rotor-Gene™ 3000 (CORBETT RESEARCH) and calculated as relative expression using Relative Expression Software Tool for Rotor-Gene© (REST-RG©). House-keeping gene was H3F3A (histon H3A). Calibrator sample was from Stratagene, and primers for house-keeping gene were from Eurogenetec and primers specific to target gene (Qiagen, Germany). Samples of cDNA and proper primers were mixed with Fast Start DNA Master SYBR Green I kit (Roche).

Immunofluorescence Staining for Flow Cytometry

On the day before the experiment 250×103 cells were cultured in Opti-MEM in sterile 25 cm² conical flasks to 60% confluence. To knock-down the Wnt1 gene, medium was removed and replaced with the transfection medium containing the siRNAs, which passed the inhibition score ranking. After 48 h the cultured cells were harvested by trypsinization and centrifuged at 2000 g, for 5 min, at 4° C. and the cells pellet was suspended in ice-cold PBS.

The cells were then fixed in 1% formaldehyde for 15 min, washed twice with PBS, suspended in ice-cold 70% ethanol and stored at −20° C. for 24 h. after this time the cells were washed twice with PBS-1% BSA and incubated for 1 h with either primary antibody anti-Wnt1 (Zymed-Invitrogen) diluted 1:250 with PBS-1% bovine serum albumin (BSA). After primary incubation the cells were washed twice with PBS-1% BSA, and incubated for 1 h with 1:500 secondary antibodies labelled with Alexa Fluor 488 (Molecular Probes, Eugene, Oreg., USA). The cells were then washed twice in PBS-1% BSA and finally incubated with a 10 μg/mL solution propidium iodide with RNase A for 15 min to counterstain the DNA. Then the cells were measured using BD FACS Calibur Flow Cytometry (Becton Dickinson, Franklin Lake, N.J., USA)

Apoptosis Analysis

For caspases 3 and 7 activation, MCF-7 cells were plated in Opti-MEM (Invitrogen) at 7×103 cells per well in 96-well plates one day before experiments. On the next day the MCF-7 cells were transfected with siRNAs sequences which passed the inhibition score ranking at a concentration of 50 nM for 48 h using Lipofectamine RNAi MAX (Invitrogen) according to manufacturer's protocol. After 12 h of siRNA exhibition, activation of caspases 3 and 7 was measured using Caspase-Glo 3/7 assay (Promega) by GloMax™ 96 Microplate Luminometer (Promega) according to manufacturer's protocol.

To analyze apoptosis cells transfected with siRNA which passed the final screening test were harvested by trypsinization and stained using an Annexin V FLUOS Staining Kit (Roche Diagnostics, Indianapolis, Ind., USA), according to the manufacture's protocol. Then stained cells were immediately analyzed by flow cytometry (FACScan; Becton Dickinson, Franklin Lake, N.J.). Early apoptotic cells with exposed phosphatidylserine but intact cell membranes bound to Annexin V-FITC but excluded propidium iodide. Cells in necrotic or late apoptotic stages were labeled with both Annexin V-FITC and propidium iodide.

Design of siRNA Sequences

One of many available algorithms may be used in the design of potent siRNA sequences. Such algorithms are commonly available in literature, such as:
1. Elbashir S M et al. (2001) Duplexes of 21-nucleotide RNAs mediate RNA interference in cultured mammalian cells. Nature. 411:494-498.
2. Elbahir S M et al. (2001). Functional anatomy of siRNAs for mediating efficient RNAi in Drosophila melanogaster embryo lysate. EMBO J. 20:6877-6888.
3. Elbashir S M et al. (2002). Analysis of gene function in somatic mammalian cells using small interfering RNAs. Methods. 26:199-213.
4. Reynolds A, Leake D, Boese Q, Scaringe S, Marshall W S, Khvorova A. Rational siRNA design for RNA interference. Nat. Biotechnol. 2004 March; 22(3):326-30.
5. Tuschl, T., Elbashir, S., Harborth, J., and Weber, K. “The siRNA User Guide”, http://www.rockefeller.edu/labheads/tuschl/sirna.html (revised May 6, 2004)
   or in the form of ready-to-use computer software:

1. http://www.ambion.com/techlib/misc/siRNA_finder.html

2. https://www.genscript.com/ssl-bin/app/rnai

3. http://wwwl.qiagen.com/Products/GeneSilencing/CustomSiRna/SiRnaDesigner.aspx

4. http://sfold.wadsworth.org/sirna.pl

The basis of these algorithms is the introduction of the mRNA or cDNA of the protein which we wish to silence.

mRNA coding the WNT-1 protein or its cDNA is easily accessible and made public (i.e. in the GENMED database: www.ncbi.nlm.nih.gov).
NCBI Reference Sequences (RefSeq) NM005430 (http://www.ncbi.nlm.nih.gov/entrez/viewer.fcgi?db=Nucleotide&dopt=GenBank&val=16936523)

The authors of the present invention have designed many potent siRNA sequences for WNT1 mRNA, which are presented in the table below (Tab. 1).

TABLE 1

| Lp. | | |
|---|---|---|
| WNT1 | | |
| se- | SENSE STRAND | ANTISENSE STRAND |
| quences | (5′ --> 3′) | (5′ --> 3′) |
| W1 | GCGUUUAUCUUCGCUAUCATT | UGAUAGCGAAGAUAAACGCTT |
| | (SEQ ID NO: 1) | (SEQ ID NO: 2) |
| W2 | CUCAUGAACCUUCACAACATT | UGUUGUGAAGGUUCAUGAGTT |
| | (SEQ ID NO: 3) | (SEQ ID NO: 4) |
| W3 | CGACCGUAUUCUCCGAGAUTT | AUCUCGGAGAAUACGGUCGTT |
| | (SEQ ID NO: 5) | (SEQ ID NO: 6) |
| W4 | UCGUCUACUUCGAGAAAUCTT | GAUUUCUCGAAGUAGACGATT |
| | (SEQ ID NO: 7) | (SEQ ID NO: 8) |
| W5 | CACUCAAGACCCGGUUAUUTT | AAUAACCGGGUCUUGAGUGTT |
| | (SEQ ID NO: 9) | (SEQ ID NO: 10) |
| W6 | CCUCCUAAGUCCCUUCCUATT | UAGGAAGGGACUUAGGAGGTT |
| | (SEQ ID NO: 11) | (SEQ ID NO: 12) |
| W7 | CACGAGUUUGGAUGUUGUAAA | UUUACAACAUCCAAACUCGUG |
| | (SEQ ID NO: 13) | (SEQ ID NO: 14) |
| W8 | UUGCACUGAAACGUGGAUACA | UGUAUCCACGUUUCAGUGCAA |
| | (SEQ ID NO: 15) | (SEQ ID NO: 16) |
| W9 | UCAGUAUUUCCUUCCACUGUA | UACAGUGGAAGGAAAUACUGA |
| | (SEQ ID NO: 17) | (SEQ ID NO: 18) |
| W10 | ACCUGCUUACAGACUCCAAGA | CUUGGAGUCUGUAAGCAGGU |
| | (SEQ ID NO: 19) | (SEQ ID NO: 20) |
| W11 | GAACCUGCUUACAGACUCCAA | UUGGAGUCUGUAAGCAGGUUC |
| | (SEQ ID NO: 21) | (SEQ ID NO: 22) |
| W12 | GCAGCUGUUGAGCCGCAAACA | UGUUUGCGGCUCAACAGCUGC |
| | (SEQ ID NO: 23) | (SEQ ID NO: 24) |
| W13 | GUACGACCGUAUUCUCCGAGA | UCUCGGAGAAUACGGUCGUAC |
| | (SEQ ID NO: 25) | (SEQ ID NO: 26) |
| W14 | ACGACCGUAUUCUCCGAGAUG | CAUCUCGGAGAAUACGGUCGU |
| | (SEQ ID NO: 27) | (SEQ ID NO: 28) |
| W15 | UACGACCGUAUUCUCCGAGAU | AUCUCGGAGAAUACGGUCGUA |
| | (SEQ ID NO: 29) | (SEQ ID NO: 30) |
| W16 | GGUUUGUCCCAGUCAGAAATT | UUUCUGACUGGGAGAAACCTA |
| | (SEQ ID NO: 31) | (SEQ ID NO: 32) |

Synthesis of siRNA

RNA synthesis was performed using the solid phase synthesis technique, using typical protocols for the synthesis of nucleic acids using derivatives of β-cyanoethyl phosphamide esters in conjunction the tert-butyldimethyl-silane protection of the 2′-OH group of ribose. Phsosphamide monomers attach to the free 5′-OH group of ribose following activation with 5-benzylmercapto-1H-tetrazole. This reaction proceeds rapidly, efficiently yielding oligomers. The oligomers formed are additionally purified using chromatographic (HPLC) or electrophoretic (PAGE) techniques. The synthesis was performed on an Applied Biosystems 962 RNA synthesizer. siRNA was produced through the gentle agitation of equimolar amounts of complementary RNA strands for 1 hour at −20° C. in 2M acetate buffer in ethanol. Such a solution was centrifuged for 15 min. and dried with 70% ethanol.

Preparation of Individual siRNA Dilutions Using a Lipid Vector

SiRNA (WNT1_{—}16) was diluted using the Hiperfect lipid vector. Hiperfect was purchased from and supplied by Qiagen.
Each siRNA dilution was prepared in a series and then an appropriate amount of HiPerFect was added.
25 nM
3 μl siRNA+1197 μl medium without serum

### 10×0.75 μl HiPerFect=7.5 μL

5 nM
200 μL 25 nM solution+800 μL medium without serum

### 8×0.75 μl HiPerFect=6 μL

1 nM
200 μL 5 nM solution+800 μL medium without serum

### 10×0.75 μl HiPerFect=7.5 μL

Transfection of siRNA:

The dilutions prepared in Example 3 were used in transfection.
siRNA transfection was performed at three concentrations: 1, 5 and 25 nm, HiPerfect: constant 0.75 microL/well. Experimental controls consisted of: a) tumour cells, b) a+HiPerfect reagent

Stages:
1. Tumour cells originating from an in vitro culture were inoculated onto a 96-well plate, at 1×10⁴ cells/well, in 100 μL. The cells were incubated for 24 hours at 37° C., in a moist environment with 5% CO₂.
2. After 24 hours of incubation, the cells were treated with an appropriate siRNA at concentrations of 1, 5 or 25 nM (final volume 100 μL), with a control consisting of cells supplemented solely with 100 μL medium or medium containing only HiPerFect. Transfection was performed according to the manufacturer's instructions found in the HiPerFect Transefection Reagent Handbook (www.qiagen.com).
3. The cells were incubated for the next 24 or 72 hours in conditions as above.

Reading of the Results

### Test Data was Recorded Using the SRB Method:

1. Following the end of incubation, 50 μl of cold 50% TCA (trichloroacetic acid) was added to each well.
2. After 60 minutes of incubation at 4° C., the cells were washed 5-times with running water.
3. After drying, each well was supplemented with 50 μL of 0.4% SRB (sulforodamine B) solution in 1% acetic acid, in order to stain the precipitated proteins.
4. Following 30 minutes of incubation at RT, the plates were rinsed 5 times with 1% acetic acid.
5. After drying, each well was supplemented with 150 μL of 10 nM TRIS buffer (tris (hydroksymethyl) aminomethane) to dissolve the dye.
6. The method was used to determine the amount of protein precipitated by the TCA. The optical density of each sample was measured spectrophotometrically at 540 nm.

The “Blank” control consisted of a solution from wells containing only culture medium. The positive control consisted of cells suspended in culture medium. The spectrophotometrically determined OD is proportional to the number of living cells in a sample. The results obtained from the measurement of the proliferation rate of individual tumour line cells treated with siRNA were collected in tables (Table 2 and Table 3)

TABLE 2
Inhibition of the proliferation of human LNCap prostate
cancer cells following 72 h of incubation with siRNA

| | siRNA concentration/inhibition of proliferation[%] | | | | | |
|---|---|---|---|---|---|---|
| | 1 nM | | 5 nM | | 25 nM | |
| SiRNA against | Average | SD | Average | SD | Average | SD |
| Wnt1 | 19.62 | 6.77 | 38.44 | 7.43 | 40.56 | 3.42 |
| Bcl2 | 9.18 | 4.16 | 24.28 | 4.14 | 30.27 | 4.88 |
| IL-6 | 6.36 | 8.99 | 12.67 | 4.38 | 7.74 | 5.95 |
| survivin | 1.29 | 1.82 | 3.97 | 5.61 | 9.25 | 13.08 |
| PSA | 4.72 | 0.08 | 10.20 | 0.40 | 5.67 | 8.02 |
| Hsp27 | 16.51 | 5.15 | 20.35 | 4.12 | 18.28 | 2.57 |
| BMX | 5.49 | 0.58 | 9.87 | 1.24 | 12.99 | 0.59 |
| MRP-1 | 16.83 | 9.05 | 19.21 | 2.67 | 23.61 | 11.37 |
| bFGF | 20.09 | 8.56 | 30.01 | 15.68 | 25.30 | 21.55 |
| DNA-PKCs | 4.60 | 3.46 | 6.91 | 5.61 | 6.02 | 8.51 |
| TIF2 | 14.41 | 16.76 | 15.51 | 17.26 | 10.59 | 16.41 |
| H01 | 7.28 | 6.97 | 17.64 | 6.17 | 13.14 | 7.16 |
| FASN | 13.51 | 14.87 | 13.65 | 13.26 | 13.26 | 13.27 |
| checkpoint | 17.33 | 15.11 | 29.05 | 17.32 | 25.67 | 22.75 |
| catenin β | 13.75 | 17.16 | 18.80 | 10.02 | 19.82 | 16.34 |

| | | |
|---|---|---|
| Control: HiPerFect | 3.58 | 4.75 |

SD—standard deviation

TABLE 3
Inhibition of the proliferation of human ASPC-1 pancreatic
cancer following 72 h of incubation with siRNA

| | siRNA concentration/inhibition of proliferation[%] | | | | | |
|---|---|---|---|---|---|---|
| | 1 nM | | 5 nM | | 25 nM | |
| SiRNA against: | Average | SD | Average | SD | Average | SD |
| H0-1 | 0.00 | 0.00 | 0.00 | 0.00 | 0.00 | 0.00 |
| AKT1 | 6.11 | 5.41 | 13.14 | 15.45 | 12.87 | 3.41 |
| Wnt1 | 10.19 | 11.95 | 16.97 | 18.72 | 16.00 | 10.85 |
| TR3 | 0.82 | 1.15 | 2.35 | 1.47 | 6.81 | 2.50 |
| Bcl-2 | 5.11 | 5.11 | 14.34 | 10.15 | 14.92 | 7.16 |
| Hsp27 | 19.04 | 20.10 | 8.87 | 11.31 | 7.52 | 4.32 |
| BMX | 5.62 | 6.10 | 12.21 | 6.26 | 9.19 | 8.05 |
| MRP-1 | 5.61 | 5.60 | 4.29 | 3.88 | 8.88 | 8.88 |
| bFGF | 7.62 | 9.06 | 8.79 | 15.15 | 12.05 | 15.02 |
| FAS | 7.38 | 4.36 | 13.01 | 12.09 | 12.21 | 12.25 |
| Survivin | 4.98 | 8.63 | 10.74 | 16.96 | 10.22 | 10.27 |
| DNMT1 | 2.08 | 2.94 | 11.16 | 8.70 | 7.39 | 0.09 |

| | | |
|---|---|---|
| Control: HiPerFect | 2.09 | 1.83 |

SD—standard deviation

From the results of the experiments on the inhibition of tumour cell proliferation, it is evident that the application of siRNA against the Wnt1 gene entails a significant inhibition of tumour cell proliferation. The values of the inhibition of proliferation are relative to control cells incubated solely in medium. Furthermore, the usage of siRNA against WNT1 resulted in a much stronger inhibitory effect on proliferation when compared to tumour cells treated solely with the Hiperfect lipid vector or the siRNA of other genes, to which anti-tumour properties are ascribed.

### Example 1

### siRNAs Against Wnt1 mRNA Inhibit Cell Growth

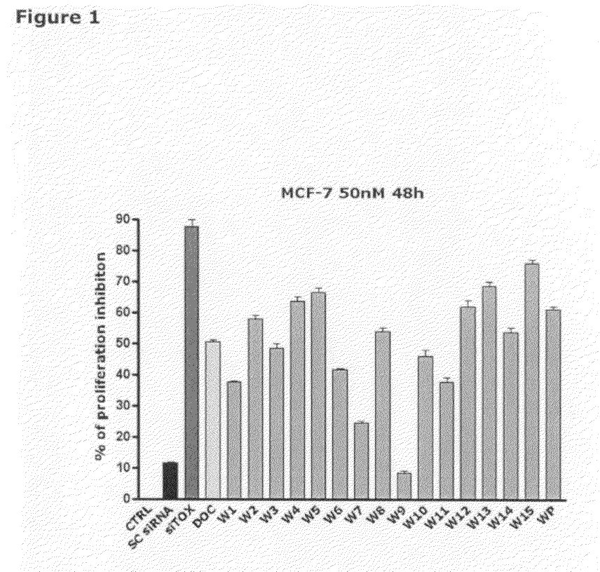

Cell proliferation of MCF-7 cells was measured over a 48 h treatment of 50 nM siRNAs sequences specific to Wnt1 gene, using MTS assay for determination of cell growth rates. The growth of cells treated with siRNA was compared to untreated cells (CTRL), cells treated with scrambled (non-coding) siRNA (SC siRNA) and to cells treated with siControl TOX (siTOX) and Docetaxel (DOC). SC siRNA and siTOX were used to determine non-specific inhibition of cell growth caused by nucleic acid chemistry or transfection reagent, and to check efficiency of transfection, respectively. Values shown on FIG. 1 indicate the percentage of proliferation rate with respect to non-transfected control cells. Non-coding siRNA had almost no effect on cell proliferation and transfection efficiency in these experiment was roughly 88%. Few of tested siRNA sequences showed great ability to reduce cell proliferation, in some cases over 50% that means higher than cytostatic drug (Docetaxel). The sequence that reached the best results on proliferation rate was W15 which inhibited proliferation by 75% relative to untreated cells and was much more effective than docetaxel and WP siRNA known from literature (He et al. 2004).

### Example 2

### siRNA is Specific and Potent in Decreasing Level of Wnt1 mRNA

Next, we measured mRNA level after MCF-7 treatment with siRNAs that passed inhibition score ranking. Reduction of mRNA levels is the most direct result of siRNA action. Thus we determined whether MCF-7 cells transfected with siRNA against Wnt1 mRNA would cause a decrease in mRNA level. Analysis was performed 48 h after transfection. Total mRNA isolation, transcription to cDNA and real-time PCR were done as described in Material and Methods. After MCF-7 cells were transfected with an siRNA sequence that targets W15 mRNA, we observed a decrease in mRNA by 61% in comparison to untreated control cells. This experiment was also a control indicating the specificity of our sequence. Additionally we performed a similar experiment with A549 cells, to check if there would be any response. It is known that there is no expression of Wnt1 in A549 cells (He et al. 2004). We observed no changes in proliferation and mRNA level 48 h after A549 cells treatment with W15 siRNA sequence. These data indicate that W15 sequence is specific and potent in decreasing mRNA level, which is a base of siRNA action.

### Example 3

### siRNA Specific to Wnt1 mRNA Decreases Protein Level

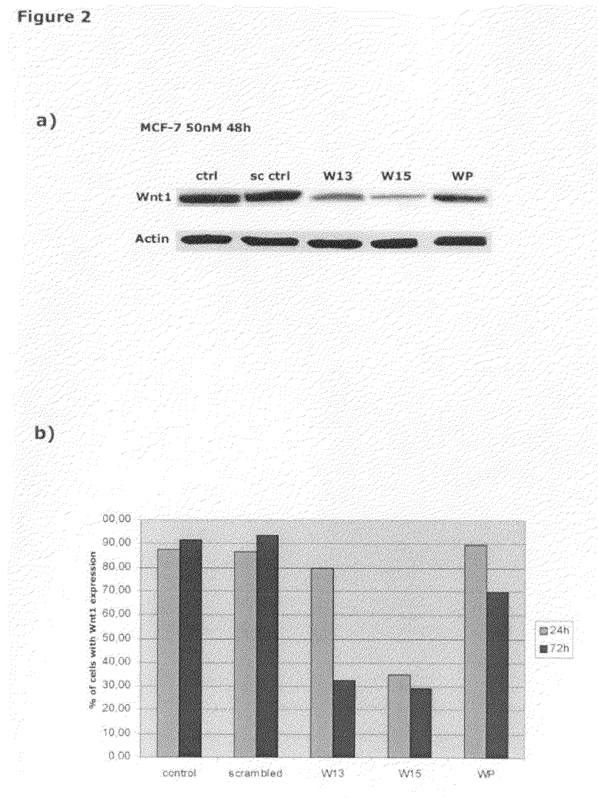

Western blotting analysis of Wnt1 level in MCF-7 cells after transfection with siRNA against Wnt1 was done (FIG. 2a). There was a decrease of Wnt1 level in cells treated with siRNA that targets the W15 mRNA sequence after 48 h and to lesser extent but also of significance in cells treated with siRNA that targets the W13 mRNA sequence relative to the control. There was a slight decrease of Wnt1 level after WP sequence treatment of MCF-7. Western blotting analysis showed an increase in the level of phosphorylated beta-catenin in MCF-7 cells after the cells were treated with siRNA that targets the Wnt1 mRNA. We observed a correlation between a decline of Wnt1 level and a decrease of c-myc and cyclin D1 levels in MCF-7 cells treated with W15 or W13 sequence. We did not observe such changes after WP sequence treatment.

These data indicate that W15 sequence against Wnt1 provides a decrease of Wnt1 level in MCF-7 cells, and it is correlated with a decline of c-myc, cyclin D1 and an increase of phosphorylated beta-catenin level.

Next, the changes in expression of Wnt1 in MCF-7 cells was measured using flow cytometry techniques after the MCF-7 cells were treated with siRNA that targets Wnt1 mRNA (FIG. 2b). 87% and 92% of control cells expressed Wnt1 after 24 h and 72 h, in turn, while only 35% and 29% of cells treated with the siRNA that targets W15 mRNA sequence had expression of Wnt1 respectively, and there were 80% and 33% cells expressing Wnt1 24 h and 72 h after transfection with an siRNA that targets the W13 mRNA sequence, while among the cells treated with an siRNA that targets the WP mRNA sequence there were 90% and 70% cells expressing Wnt1 respectively. This analysis shows that siRNA against Wnt1 induces protein level decrease.

### Example 4

### siRNA Against Wnt1 Induced Apoptosis but not Necrosis

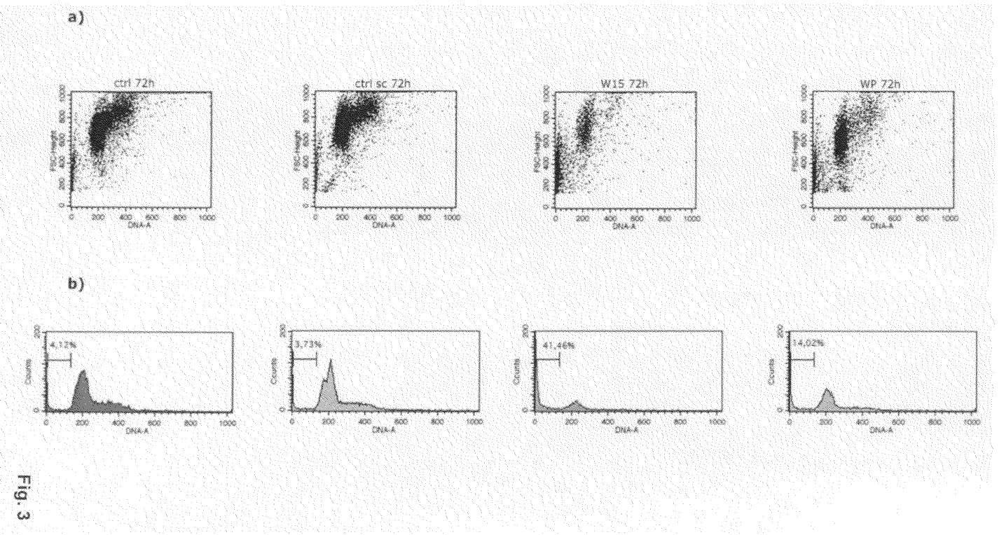

Analysis of cell cycle of MCF-7 cells treated with siRNA against Wnt1 mRNA was done using flow cytometry techniques (FIG. 3). After 72 h we observed 41% of dead cells in comparison to control (4%) and the cells treated with an siRNA that targets the WP mRNA sequence (14%). These data showed that transfection of MCF-7 cells with siRNA that targets the Wnt1 mRNA increased the MCF-7 cell death.

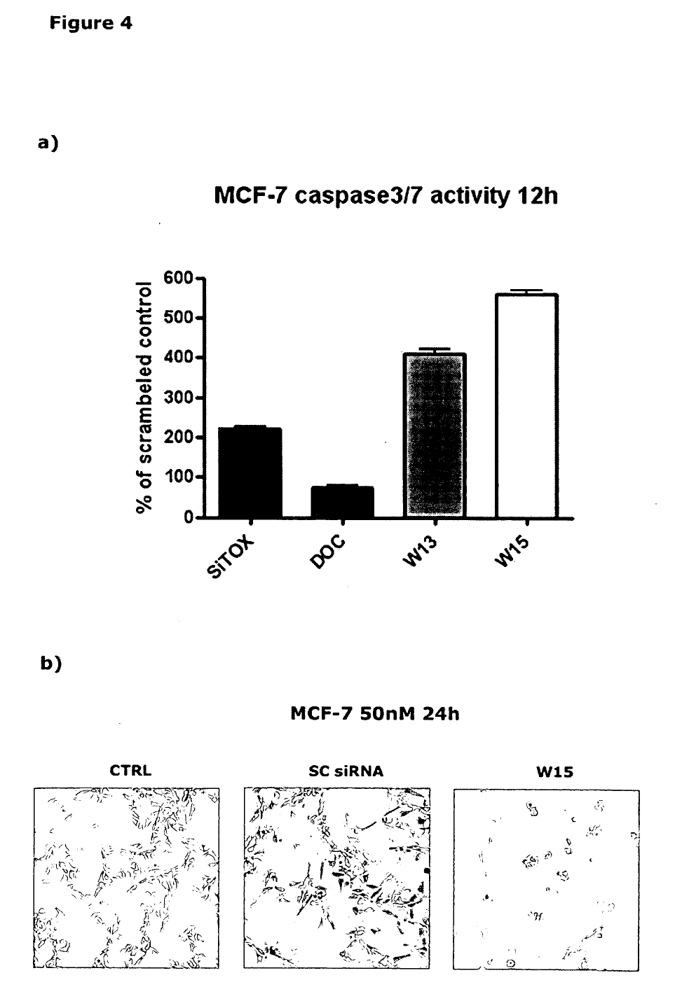

To verify what kind of cell death is triggered by siRNA treatment we performed caspases activation assay. The results obtained in this assay are presented as inhibition of proliferation in comparison to control. We observed that after treatment of MCF-7 cells with W15 sequence there was at least fivefold increase in activation of caspases 3 and 7, and after W13 sequence treatment it was around fourfold increase while after treatment with cytotoxic docetaxel it was only about twofold increase (FIG. 4a). This results were confirmed by morphological changes of MCF-7 cells after treatment with W15 sequence (FIG. 4b). These results show that the siRNA sequence that targets the W15 mRNA sequence induces apoptosis in MCF-7 cells.

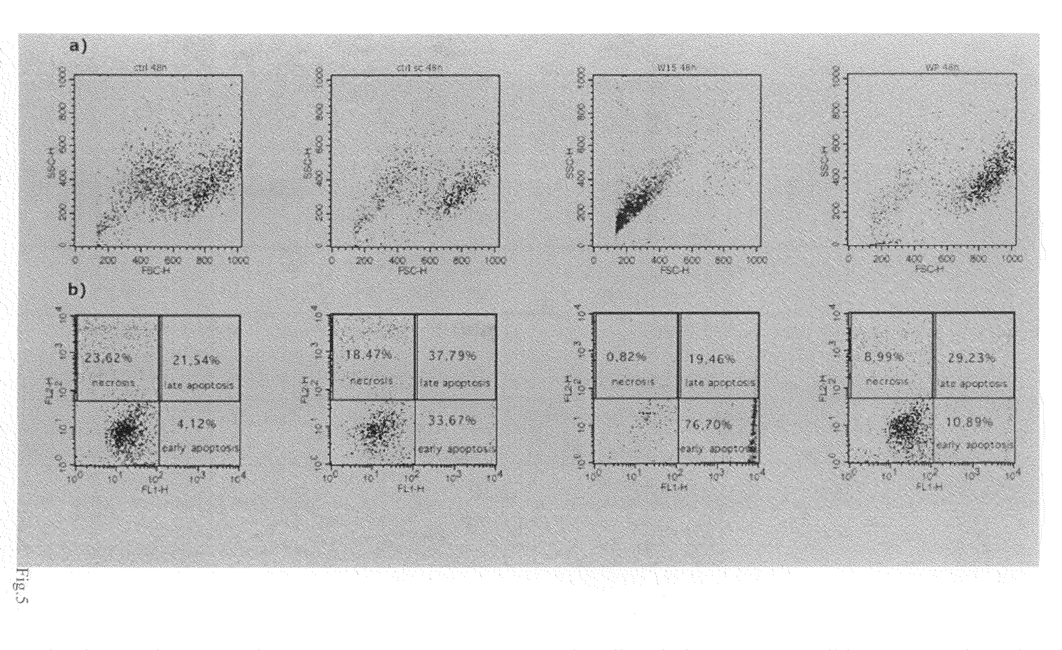

We then determined the number of apoptotic cells, of necrotic cells and of viable cells. Analysis of apoptosis using Annexin V (AV) and propidium iodide (PI) double staining was performed. Double negative are viable cells. AV positive and PI negative are cells in early phase of apoptosis, while AV positive and PI positive are cells in a late phase of apoptosis. Necrotic cells are AV negative and PI positive (FIG. 5).

### Example 5

### Decrease of Protein Level Induced by siRNA Specific to Wnt1 Provokes Apoptosis

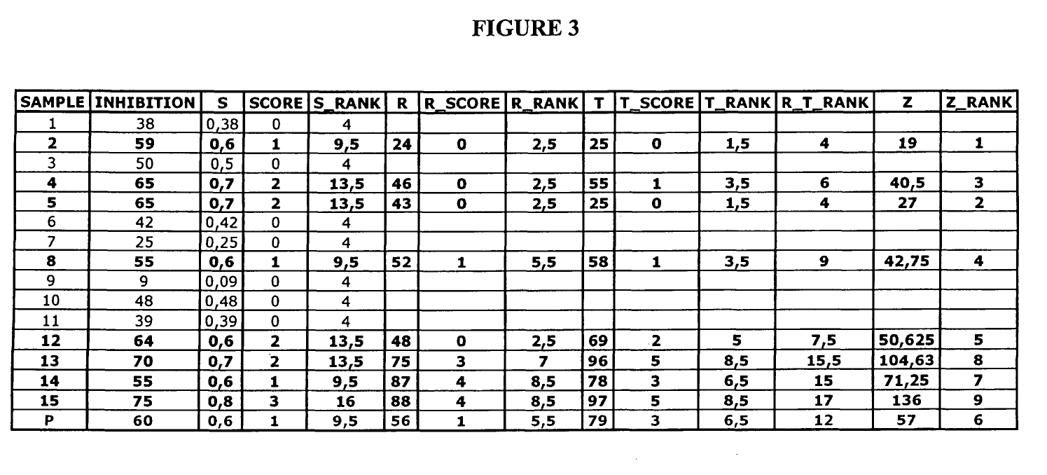

Flow cytometry technique was used to verify if apoptosis was triggered by of the reduction in the level of Wnt1 in MCF-7 cells transfected with siRNA that targets the Wnt1 mRNA. (FIG. 6). Among control cells that expressed Wnt1, 87% of them were alive, while only 9% of the cells with no detectable Wnt1 expression were alive and 4% cells were dead with no Wnt1 expression after 24 h of growth. After 72 h of cell growth 90% of the cells were alive with Wnt1 expression, 4% of the cells were alive with no Wnt1 expression and 3% dead cells with no Wnt1 expression was observed. In turn among cells treated with siRNA specific to Wnt1 there were 34% alive cells with Wnt1 expression, while 24% cells were alive with no Wnt1 expression and 41% cells were dead with no Wnt1 expression after 24 h. There were 25% alive cells with Wnt1 expression, while 3% cells were alive with no Wnt1 expression and 68% cells were dead with no Wnt1 expression after 72 h. We did not observed such changes after WP sequence treatment. These data indicate that apoptosis is triggered by decrease of Wnt1 level induced by siRNA specific for the Wnt1 mRNA.

The teachings of all of the references including websites cited herein are incorporated in their entirety by reference.

| The cDNA sequence of the WNT-1 gene (Accesion No. NM005430) | |
|---|---|
| (SEQ ID NO: 33) | |
| 1 | gcggtgccgc ccgccgtggc cgcctcagcc caccagccgg gaccgcgagc catgctgtcc |
| 61 | gccgcccgcc cccagggttg ttaaagccag actgcgaact ctcgccactg ccgccaccgc |
| 121 | cgcgtcccgt cccaccgtcg cgggcaacaa ccaaagtcgc cgcaactgca gcacagagcg |
| 181 | ggcaaagcca ggcaggccat ggggctctgg gcgctgttgc ctggctgggt ttctgctacg |
| 241 | ctgctgctgg cgctggccgc tctgcccgca gccctggctg ccaacagcag tggccgatgg |
| 301 | tggggtattg tgaacgtagc ctcctccacg aacctgctta cagactccaa gagtctgcaa |
| 361 | ctggtactcg agcccagtct gcagctgttg agccgcaaac agcggcgtct gatacgccaa |
| 421 | aatccgggga tcctgcacag cgtgagtggg gggctgcaga gtgccgtgcg cgagtgcaag |
| 481 | tggcagttcc ggaatcgccg ctggaactgt cccactgctc cagggcccca cctcttcggc |
| 541 | aagatcgtca accgaggctg tcgagaaacg gcgtttatct tcgctatcac ctccgccggg |
| 601 | gtcacccatt cggtggcgcg ctcctgctca gaaggttcca tcgaatcctg cacgtgtgac |
| 661 | taccggcggc gcggccccgg gggccccgac tggcactggq ggggctgcag cgacaacatt |
| 721 | gacttcggcc gcctcttcgg ccgggagttc gtggactccg gggagaaggg gcgggacctg |
| 781 | cgcttcctca tgaaccttca caacaacgag gcaggccgta cgaccgtatt ctccgagatg |
| 841 | cgccaggagt gcaagtgcca cgggatgtcc ggctcatgca cggtgcgcac gtgctggatg |
| 901 | cggctgccca cgctgcgcgc cgtgggcgat gtgctgcgcg accgcttcga cggcgcctcg |
| 961 | cgcgtcctgt acggcaaccg cggcagcaac cgcgcttcgc gagcggagct gctgcgcctg |
| 1021 | gagccggaag acccggccca caaaccgccc tccccccacg acctcgtcta cttcgagaaa |
| 1081 | tcgcccaact tctgcacgta cagcggacgc ctgggcacag caggcacggc agggcgcgcc |
| 1141 | tgtaacagct cgtcgcccgc gctggacggc tgcgagctgc tctgctgcgg caggggccac |
| 1201 | cgcacgcgca cgcagcgcgt caccgagcgc tgcaactgca ccttccactg gtgctgccac |
| 1261 | gtcagctgcc gcaactgcac gcacacgcgc gtactgcacg agtgtctgtg aggcgctgcg |
| 1321 | cggactcgcc cccaggaaac gctctcctcg agccctcccc caaacagact cgctagcact |
| 1381 | caagacccgg ttattcgccc acccgagtac ctccagtcac actccccgcg gttcatacgc |
| 1441 | atcccatctc tcccacttcc tcctacctgg ggactcctca aaccacttgc ctggggcggc |
| 1501 | atgaaccctc ttgccatcct gatggacctg ccccggacct acctccctcc ctctccgcgg |
| 1561 | gagacccctt gttgcactgc cccctgcttg gccaggaggt gagagaagga tgggtcccct |
| 1621 | ccgccatggg gtcggctcct gatggtgtca ttctgcctgc tccatcgcgc cagcgacctc |
| 1681 | tctgcctctc ttcttcccct ttgtcctgcg ttttctccgg gtcctcctaa gtcccttcct |
| 1741 | attctcctgc catgggtgca gaccctgaac ccacacctgg gcatcagggc ctttctcctc |
| 1801 | cccacctgta gctgaagcag gaggttacag ggcaaaaggg cagctgtgat gatgtggaaa |
| 1861 | tgaggttggg ggaaccagca gaaatgcccc cattctccca gtctctgtcg tggagccatt |
| 1921 | gaacagctgt gagccatgcc tccctgggcc acctcctacc ccttcctgtc ctgcctcctc |
| 1981 | atcagtgtgt aaataatttg cactgaaacg tggatacaga gccacgagtt tggatgttgt |
| 2041 | aaataaaact atttattgtg ctgggtccca gcctggtttg caaagaccac ctccaaccca |
| 2101 | acccaatccc tctccactct tctctccttt ctccctgcag ccttttctgg tccctcttct |
| 2161 | ctcctcagtt tctcaaagat gcgtttgcct cctggaatca gtatttcctt ccactgtagc |
| 2221 | tattagcggc tcctcgcccc caccagtgta gcatcttcct ctgcagaata aaatctctat |
| 2281 | ttttatcgat gacttggtgg cttttccttg aatccagaac acaaccttgt ttgtggtgtc |
| 2341 | ccctatcctc cccttttacc actcccag |

1. A method for obtaining an oligonucleotide useful as an effective anticancer agent comprising:
obtaining a known sequence of an mRNA encoded by a gene involved in carcinogenesis from a database,
generating siRNAs sequences against the chosen mRNA sequence are generated in silico using known algorithms,
ranking the siRNA sequences according to total filtering score,
choosing oligonucleotides comprised of no more than 30 bp, preferably 21 to 23 bp,
synthesizing said olignucleotides,
screening the synthesized oligonucleotides for inhibition of proliferation of cancerous cells upon transfection of the oligonucleotides into said cancerous cells,
screening the synthesized oligonucleotides for an ability of said oligonucleotides to induce a reduction of a targeted mRNA upon transfection of the oligonucleotides into said cancerous cells level is performed,
screening the synthesized oligonucleotides for an ability of said oligonucleotides to decrease levels of a protein associated with cancer upon transfection of said oligonucleotides into cancerous cells,
screening the synthesized oligonucleotides by factor z wherein,:
$$z = ( \frac{b + c}{2} ) * a$$
where:
a—rank by the inhibition score,
b—rank by the decrease in mRNA level score,
c—rank by the decrease in protein level score,
analyzing cell death mechanism for the oligonucleotides with z factor greater or equal to 50% of the best sequence and a oligonucleotide providing at least 50% level of cancer cell apoptosis is selected as the oligonucleotide useful as effective an anticancer agent.

2. The method according to claim 1, characterised in that the total filtering score is evaluated on the base of at least one of the following parameters: frequency among algorithms, single stranded region probability, complementary to other mRNA sequences, free energy of the antisense strand 5′ end, free energy of the antisense strand 3′ end, free energy at a 10 position of an antisense strand, GC content.

3. The method according to claim 1, characterised in that the inhibition score for each oligonucleotide is evaluated by factor s:
$$s = 1 - ( \frac{Rs - Rm}{Rc - Rm} ) ,$$
where:
Rs—the result of a measurement of a probe with siRNA,
Rm—the result of a measurement of a blank probe,
Rc—the result of a measurement of a probe with control,
wherein inhibition score is:
0 if s lower than 0.50,
1 if s value 0.51-0.60,
2 if s value 0.61-0.70,
3 if s value 0.71-0.80,
4 if s value 0.81-0.90,
5 if s value 0.91-1.00,
and the oligonucleotides are ranked by the obtained scores.

4. The method according to claim 1, characterised in that a decrease in mRNA level score for each sequence is evaluated by factor r wherein:
$$r = 100 - ( \frac{Es}{Ec} * 100 ) .$$
where:
Es—relative expression of target gene in probe with siRNA
Ec—relative expression of target gene in probe with control
wherein decrease in mRNA level score is:
0 if r lower than 50,
1 if r value 51-60,
2 if r value 61-70,
3 if r value 71-80,
4 if r value 81-90,
5 if r value 91-100,
and the oligonucleotides are ranked by the obtained scores.

5. The method according to claim 1, characterised in that a decrease in protein level score for each sequence was is evaluated by factor t:
$$t = 100 - ( \frac{Ps}{Pc} * 100 )$$
where:
Ps—protein level in probe with siRNA
Pc—protein level in probe with control
wherein decrease in protein level score is:
0 if t lower than 50,
1 if t value 51-60,
2 if t value 61-70,
3 if t value 71-80,
4 if t value 81-90,
5 if t value 91-100,
and the oligonucleotides are ranked by the obtained scores.

6. A double helix oligonucleotide providing at least 50% level of cancer cell apoptosis obtained by a process comprising:
obtaining a known sequence of an mRNA encoded by a gene involved in carcinogenesis from a database,
generating siRNAs sequences against the chosen mRNA sequence in silico using known algorithms,
ranking the siRNA sequences according to a total filtering score,
choosing oligonucleotides comprised of no more than 30 base pairs (bP), preferably 21 to 23 bp,
synthesizing said oligonucleotidess,
screening the synthesized oligonucleotides for inhibition of proliferation of cancerous cells upon transfection of the oligonucleotides into said cancerous cells,
screening the synthesized oligonucleotides for an ability of said oligonucleotides to induce a reduction of a targeted mRNA upon transfection of the oligonucleotides into said cancerous cells,
screening the synthesized oligonucleotides for an ability of said oligonucleotides to decrease levels of a protein associated with cancer upon transfection of said oligonucleotides into cancerous cells,
screening the synthesized oligonucleotides by factor z wherein,
$$z = ( \frac{b + c}{2} ) * a$$
where:
a—rank by the inhibition score,
b—rank by the decrease in mRNA level score,
c—rank by the decrease in protein level score,
analyzing cell death mechanism for the oligonucleotides with z factor greater or equal to 50% of the best sequence and the oligonucleotide providing at least 50% level of cancer cell apoptosis is selected as the oligonucleotide useful as effective an anticancer agent.

7. The oligonucleotide according to claim 6 further obtained by a method characterised in that the total filtering score is evaluated on the basis of at least one of the following parameters: frequency among algorithms, single stranded region probability, complementary to other mRNA sequences, free energy of the antisense strand 5′ end, free energy of the antisense strand 3′ end, free energy at 10 position of the antisense strand, GC content.

8. The double helical oligonucleotide of claim 6 further characterized characterised in that an inhibition score for each oligonucleotide is evaluated by factor s:
$$s = 1 - ( \frac{Rs - Rm}{Rc - Rm} ) ,$$
where:
Rs—the result of a measurement of a probe with siRNA,
Rm—the result of a measurement of a blank probe,
Rc—the result of a measurement of a probe with control,
wherein inhibition score is:
0 if s lower than 0.50,
1 if s value 0.51-0.60,
2 if s value 0.61-0.70,
3 if s value 0.71-0.80,
4 if s value 0.81-0.90,
5 if s value 0.91-1.00,
and the oligonucleotides are ranked by the obtained scores.

9. A double helical oligonucleotide of claim 6 further characterized by being produced by a process characterised in that a decrease in mRNA level score for each sequence is evaluated by factor r wherein:
$$r = 100 - ( \frac{Es}{Ec} * 100 )$$
where:
Es—relative expression of target gene in probe with siRNA
Ec—relative expression of target gene in probe with control
wherein decrease in mRNA level score is:
0 if r lower than 50,
1 if r value 51-60,
2 if r value 61-70,
3 if r value 71-80,
4 if r value 81-90,
5 if r value 91-100,
and the oligonucleotides are ranked by the obtained scores.

10. A double helical oligonucleotide of claim 6 further characterized in that the oligonucleotide is produced by a method characterised in that a decrease in protein level score for each sequence was evaluated by factor t:
$$t = 100 - ( \frac{Ps}{Pc} * 100 )$$
where:
Ps—protein level in probe with siRNA
Pc—protein level in probe with control
wherein decrease in protein level score is:
0 if t lower than 50,
1 if t value 51-60,
2 if t value 61-70,
3 if t value 71-80,
4 if t value 81-90,
5 if t value 91-100,
and the oligonucleotides are ranked by the obtained scores.

11. An siRNA molecule for inhibition of Wnt mRNA comprised of 15 to 30 consecutive nucleotides that targets an mRNA sequence of a Wnt1 cDNA presented in FIG. 1.

12. An siRNA of claim 11 wherein the siRNA sequence contains a sense and antisense sequence selected from the group consisting SEQ NOs:1-32.

13. An siRNA molecule of claim 11 wherein the siRNA contains one or more chemical modifications.

14. A method for treating cancer associated with a Wnt gene comprising bringing an siRNA that targets an mRNA expressed by the Wnt gene into contact with a cancerous cell that expresses said Wnt gene.

15. The method of claim 14 wherein the siRNA has a sense strand and an antisense strand selected from the group consisting of SEQ ID NOs:1-32.

16. The method of claim 14 wherein the cancerous cell is a prostate or pancreatic cancerous cell.


SEQUENCE LISTING (33 sequences)

SEQ ID NO: 1 - DNA, 21 bp, Homo sapiens
gcguuuaucuucgcuaucatt

SEQ ID NO: 2 - DNA, 21 bp, Homo sapiens
ugauagcgaagauaaacgctt

SEQ ID NO: 3 - DNA, 21 bp, Homo sapiens
cucaugaaccuucacaacatt

SEQ ID NO: 4 - DNA, 21 bp, Homo sapiens
uguugugaagguucaugagtt

SEQ ID NO: 5 - DNA, 21 bp, Homo sapiens
cgaccguauucuccgagautt

SEQ ID NO: 6 - DNA, 21 bp, Homo sapiens
aucucggagaauacggucgtt

SEQ ID NO: 7 - DNA, 21 bp, Homo sapiens
ucgucuacuucgagaaauctt

SEQ ID NO: 8 - DNA, 21 bp, Homo sapiens
gauuucucgaaguagacgatt

SEQ ID NO: 9 - DNA, 21 bp, Homo sapiens
cacucaagacccgguuauutt

SEQ ID NO: 10 - DNA, 21 bp, Homo sapiens
aauaaccgggucuugagugtt

SEQ ID NO: 11 - DNA, 21 bp, Homo sapiens
ccuccuaagucccuuccuatt

SEQ ID NO: 12 - DNA, 21 bp, Homo sapiens
uaggaagggacuuaggaggtt

SEQ ID NO: 13 - RNA, 21 nt, Homo sapiens
cacgaguuuggauguuguaaa

SEQ ID NO: 14 - RNA, 21 nt, Homo sapiens
uuuacaacauccaaacucgug

SEQ ID NO: 15 - RNA, 21 nt, Homo sapiens
uugcacugaaacguggauaca

SEQ ID NO: 16 - RNA, 21 nt, Homo sapiens
uguauccacguuucagugcaa

SEQ ID NO: 17 - RNA, 21 nt, Homo sapiens
ucaguauuuccuuccacugua

SEQ ID NO: 18 - RNA, 21 nt, Homo sapiens
uacaguggaaggaaauacuga

SEQ ID NO: 19 - RNA, 21 nt, Homo sapiens
accugcuuacagacuccaaga

SEQ ID NO: 20 - RNA, 20 nt, Homo sapiens
cuuggagucuguaagcaggu

SEQ ID NO: 21 - RNA, 21 nt, Homo sapiens
gaaccugcuuacagacuccaa

SEQ ID NO: 22 - RNA, 21 nt, Homo sapiens
uuggagucuguaagcagguuc

SEQ ID NO: 23 - RNA, 21 nt, Homo sapiens
gcagcuguugagccgcaaaca

SEQ ID NO: 24 - RNA, 21 nt, Homo sapiens
uguuugcggcucaacagcugc

SEQ ID NO: 25 - RNA, 21 nt, Homo sapiens
guacgaccguauucuccgaga

SEQ ID NO: 26 - RNA, 21 nt, Homo sapiens
ucucggagaauacggucguac

SEQ ID NO: 27 - RNA, 21 nt, Homo sapiens
acgaccguauucuccgagaug

SEQ ID NO: 28 - RNA, 21 nt, Homo sapiens
caucucggagaauacggucgu

SEQ ID NO: 29 - RNA, 21 nt, Homo sapiens
uacgaccguauucuccgagau

SEQ ID NO: 30 - RNA, 21 nt, Homo sapiens
aucucggagaauacggucgua

SEQ ID NO: 31 - DNA, 21 bp, Homo sapiens
gguuugucccagucagaaatt

SEQ ID NO: 32 - DNA, 21 bp, Homo sapiens
uuucugacugggacaaaccta

SEQ ID NO: 33 - DNA, 2368 bp, Homo sapiens
gcggtgccgcccgccgtggccgcctcagcccaccagccgggaccgcgagccatgctgtccgccgcccgcccccagggttgttaaagccagactgcgaactctcgccactgccgccaccgccgcgtcccgtcccaccgtcgcgggcaacaaccaaagtcgccgcaactgcagcacagagcgggcaaagccaggcaggccatggggctctgggcgctgttgcctggctgggtttctgctacgctgctgctggcgctggccgctctgcccgcagccctggctgccaacagcagtggccgatggtggggtattgtgaacgtagcctcctccacgaacctgcttacagactccaagagtctgcaactggtactcgagcccagtctgcagctgttgagccgcaaacagcggcgtctgatacgccaaaatccggggatcctgcacagcgtgagtggggggctgcagagtgccgtgcgcgagtgcaagtggcagttccggaatcgccgctggaactgtcccactgctccagggccccacctcttcggcaagatcgtcaaccgaggctgtcgagaaacggcgtttatcttcgctatcacctccgccggggtcacccattcggtggcgcgctcctgctcagaaggttccatcgaatcctgcacgtgtgactaccggcggcgcggccccgggggccccgactggcactgggggggctgcagcgacaacattgacttcggccgcctcttcggccgggagttcgtggactccggggagaaggggcgggacctgcgcttcctcatgaaccttcacaacaacgaggcaggccgtacgaccgtattctccgagatgcgccaggagtgcaagtgccacgggatgtccggctcatgcacggtgcgcacgtgctggatgcggctgcccacgctgcgcgccgtgggcgatgtgctgcgcgaccgcttcgacggcgcctcgcgcgtcctgtacggcaaccgcggcagcaaccgcgcttcgcgagcggagctgctgcgcctggagccggaagacccggcccacaaaccgccctccccccacgacctcgtctacttcgagaaatcgcccaacttctgcacgtacagcggacgcctgggcacagcaggcacggcagggcgcgcctgtaacagctcgtcgcccgcgctggacggctgcgagctgctctgctgcggcaggggccaccgcacgcgcacgcagcgcgtcaccgagcgctgcaactgcaccttccactggtgctgccacgtcagctgccgcaactgcacgcacacgcgcgtactgcacgagtgtctgtgaggcgctgcgcggactcgcccccaggaaacgctctcctcgagccctcccccaaacagactcgctagcactcaagacccggttattcgcccacccgagtacctccagtcacactccccgcggttcatacgcatcccatctctcccacttcctcctacctggggactcctcaaaccacttgcctggggcggcatgaaccctcttgccatcctgatggacctgccccggacctacctccctccctctccgcgggagaccccttgttgcactgccccctgcttggccaggaggtgagagaaggatgggtcccctccgccatggggtcggctcctgatggtgtcattctgcctgctccatcgcgccagcgacctctctgcctctcttcttcccctttgtcctgcgttttctccgggtcctcctaagtcccttcctattctcctgccatgggtgcagaccctgaacccacacctgggcatcagggcctttctcctccccacctgtagctgaagcaggaggttacagggcaaaagggcagctgtgatgatgtggaaatgaggttgggggaaccagcagaaatgcccccattctcccagtctctgtcgtggagccattgaacagctgtgagccatgcctccctgggccacctcctaccccttcctgtcctgcctcctcatcagtgtgtaaataatttgcactgaaacgtggatacagagccacgagtttggatgttgtaaataaaactatttattgtgctgggtcccagcctggtttgcaaagaccacctccaacccaacccaatccctctccactcttctctcctttctccctgcagccttttctggtccctcttctctcctcagtttctcaaagatgcgtttgcctcctggaatcagtatttccttccactgtagctattagcggctcctcgcccccaccagtgtagcatcttcctctgcagaataaaatctctatttttatcgatgacttggtggcttttccttgaatccagaacacaaccttgtttgtggtgtcccctatcctccccttttaccactcccag